(19) United States
(12) Patent Application Publication
(10) Pub. No.: US 2008/0269884 A1
(43) Pub. Date: Oct. 30, 2008

(54) GRADUATED BLUE FILTERING INTRAOCULAR LENS

(75) Inventor: Stephen J. Vannoy, Southlake, TX (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: Alcon, Inc., Fort Worth, TX (US)

(21) Appl. No.: 12/112,267

(22) Filed: Apr. 30, 2008

Related U.S. Application Data
(60) Provisional application No. 60/914,996, filed on Apr. 30, 2007.

(51) Int. Cl. A61F 2/16

(52) U.S. Cl. 623/6.17

(57) ABSTRACT

Devices and methods utilizing novel intraocular lens (IOL) designs are discussed herein. One aspect relates to IOLs having an optic with non-uniform light transmissivity. For example, the optic of the IOL can include a central region having a reduced light transmissivity relative to another portion of the optic. In addition, or alternatively, the optic can have a peripheral region having reduced light transmissivity. Such IOLs can potentially be utilized to alter the light distribution impinging on a subject's retina, which can be tailored to specific lighting situations such as bright and dim light conditions. Such IOLs can also, or alternatively, be used to help alleviate the perception of dark shadows known as negative dysphotopsia. Other aspects and features of IOLs, and methods, are also discussed.

### CROSS REFERENCE TO RELATED APPLICATIONS

The present application claims the benefit of a provisional application bearing Ser. No. 60/914,996, filed Apr. 30, 2007, entitled “Graduated Blue-Filtering Intraocular Lens.”

This application is also related to the following commonly owned patent applications: “Intraocular Lens with Asymmetric Haptics” (Attorney Docket No. 3227); “Intraocular Lens with Asymmetric Optics” (Attorney Docket No. 3360), bearing Ser. No. 11/742,035; “Haptic Junction Designs To Reduce Negative Dysphotopsia” (Attorney Docket No. 3344); “Intraocular Lens with Peripheral Region Designed to Reduce Negative Dysphotopsia” (Attorney Docket No. 2817), bearing Ser. No. 11/742,041; “IOL Peripheral Surface Designs To Reduce Negative Dysphotopsia” (Attorney Docket No. 3345); “Intraocular Lens with Edge Modification” (Attorney Docket No. 3225), bearing Ser. No. 11/742,202; and “A New Ocular Implant to Correct Dysphotopsia, Glare, Halo, and Dark Shadow Type Phenomena” (Attorney Docket No. 3226), bearing Ser. No. 11/742,320. Each application in this paragraph was filed on Apr. 30, 2007.

All of aforementioned applications are incorporated herein by reference in their entirety.

### BACKGROUND

The present invention relates generally to intraocular lenses (IOLs), and particularly to IOLs that provide spatially and/or spectrally non-uniform filtering of incident light to protect the retina from potentially harmful rays and to inhibit the perception of visual artifacts in the peripheral visual field.

The optical power of the eye is determined by the optical power of the cornea and that of the natural crystalline lens, with the lens providing about a third of the eye's total optical power. The process of aging as well as certain diseases, such as diabetes, can cause clouding of the natural lens, a condition commonly known as cataract, which can adversely affect a patient's vision.

Intraocular lenses (IOLs) are routinely employed to replace such a clouded natural lens. Although such IOLs can substantially restore the quality of a patient's vision, such vision is not necessarily perfect. For instance, depending upon the general lighting to which a subject's eye is exposed, an IOL may not allow an optimal amount of light into the eye. Conventional IOLs oftentimes utilize an optic having essentially constant light transmissivity over the entire optic. Using a constant high light transmissivity can allow high intensity light into the eye, which can be bothersome (e.g., on sunny days). Using a constant low light transmissivity, however, can result in visual perception difficulties in low light situations.

Accordingly, there is a need for enhanced IOLs, and particularly for IOLs and methods that can address some of the issues related to adjusting for various lighting situations.

### SUMMARY

Embodiments of the present invention are directed to devices and methods related to intraocular lenes (IOLs) in which an optic of the IOL can have a non-uniform light transmissivity across the optic. By providing different portions of the optic with different light transmissivities, the amount of light entering the eye can be effectively altered, potentially accounting for varying light situations. Furthermore, tailoring the light transmissivity in a peripheral region of the optic can potentially act to inhibit dysphotopsia, e.g., by redirecting light rays that can result in secondary image formation on the retina. Graduated changes in light can also, or alternatively, help to avoid sharp contrast changes, which can also be beneficial.

One aspect is drawn to an intraocular lens (IOL) having an optic for implantation in a subject's eye. The optic can exhibit non-uniform light transmissivity for one or more wavelengths (e.g., the blue portion of the visible spectrum) over at least a portion of the optic so as to inhibit perception of visual artifacts in a peripheral visual field of the subject's eye. For instance, the optic can include one or more dyes adapted such that a portion of the optic has at least one selected light transmissivity. The optic can include a peripheral region that exhibits reduced light transmissivity, in at least a segment thereof (e.g., a segment positioned on the nasal side of the eye when the IOL is implanted), relative to another portion of the optic. In one embodiment, the non-uniform light transmissivity of the optic is characterized by a center region having a higher light transmissivity relative to a peripheral region. For example, the light transmissivity of at least one wavelength in the center region of the optic can be no more than about 50 percent, and the corresponding light transmissivity in the peripheral region can be no more than about 10 percent.

In another embodiment, the non-uniform light transmissivity of an optic can be characterized by either a linear or non-linear gradient in any of the center region and the peripheral region. For example, the light transmissivity in the center region of the optic can be characterized by filtering at least one wavelength below about 500 nanometers, and/or the light transmissivity in the peripheral region of the optic can be characterized by filtering at least one wavelength below about 700 nanometers.

In other embodiments, the non-uniform light transmissivity of an optic can be characterized by an increase in light transmissivity (e.g., by a linear or non-linear gradient) from a center of the optic to an intermediate region (intermediacy) of the optic. As well, the optic can also, or alternatively, exhibit a decrease in light transmissivity (e.g., by a linear or non-linear gradient) from the intermediacy of the optic to the periphery of the optic. For example, the light transmissivity from the center to the intermediacy for at least one wavelength can be in relation to a wavelength range from about 400 nm to about 500 nm. As well, the light transmissivity from the intermediary to the periphery for at least one wavelength can be in relation to the visible spectrum. The light transmissivities in either or both the first gradient and second gradients can be characterized by at least one value of less than about 50 percent. Light transmissivities in the intermediacy of the optic can be characterized by at least one value greater than about 90 percent.

In another aspect, an IOL can include an optic for implantation in a subject's eye in which the optic exhibits non-uniform light transmissivity (e.g., a blue portion of the visible electromagnetic spectrum) such that light transmissivity in an inner region of the optic is greater than light transmissivity in an outer region of the optic. At least a section of the outer region of the optic with reduced light transmissivity relative to another portion of the optic can be positioned on the nasal side of the eye once the IOL is implanted in the eye. The light transmissivities of the inner and outer regions can each be independently characterized by a linear or non-linear gradient. The inner region's light transmissivity can be characterized by filtering at least one wavelengths below about 500 nanometers, and the light transmissivity in the outer region can be characterized by filtering at least one wavelength below about 700 nanometers.

Another aspect is directed to an IOL that includes an optic for implantation in a subject's eye, where the optic exhibits non-uniform light transmissivity (e.g., a blue portion of the visible electromagnetic spectrum) such that light transmissivity in an inner region of the optic is less than light transmissivity in an intermediate region of the optic, and light transmissivity in an outer region of the optic is less than light transmissivity in the intermediate region. At least a segment of the outer region of the optic with reduced light transmissivity relative to another portion of the optic can be positioned on the nasal side of the eye once the IOL is implanted. The optic can include one or more gradients in light transmissivity, which can be either linear or non-linear. A first gradient can be characterized by filtering one or more wavelengths below about 500 nanometers, and a second gradient can be characterized by filtering one or more wavelengths below about 700 nanometers.

Yet another aspect is directed to an IOL including an optic disposed about an optical axis. The optic can exhibit light transmissivity that is symmetric about the optical axis and radially non-uniform relative to the optical axis, the radial non-uniformity being adapted to inhibit perception of peripheral visual artifacts. For instance, the radial non-uniformity can be characterized by a center region having higher light transmissivity than a peripheral region of the optic. Embodiments of such an IOL can include one or more additional features with respect to the various aspects discussed above.

For any of the IOLs summarized herein, the optic can optionally include at least one haptic for attaching the IOL to a patient. The haptic(s) can be formed integrally with the optic (e.g., milled from a piece of material such as polymethylmethacrylate), or formed from separate pieces. In some instances, the haptic can also exhibit light transmissivity, e.g., a non-uniform light transmissivity. For example, the transmissivity for one or more wavelengths of light can be continuous with that of the portion of the optic to which the haptic is coupled. This can allow for more consistent light filtering of the IOL. The non-uniform light transmissivity in a haptic can have any of the filtering features discussed with respect to optics. In some instances, the haptic closest to the nasal side exhibits non-uniform light transmissivity, while the other haptic(s) may or may not exhibit such properties.

A method for inhibiting dysphotopsia in a patient having an implanted IOL is encompassed in another aspect of the invention. Peripheral light rays (e.g., entering from a temporal side of the eye) intercepted by the IOL can be directed such as to inhibit perception of visual artifacts in a peripheral visual field of an eye of the patient. The IOL can have non-uniform light transmissivity for at least one visible wavelength. The light transmissivities between a peripheral region of the IOL and another portion of the IOL can be different, e.g., the peripheral region having a lower light transmissivity.

### BRIEF DESCRIPTION OF THE DRAWINGS

FIG. 1A is a schematic cross-sectional view of an IOL according to one embodiment of the invention,

FIG. 1B is a top view of the IOL of FIG. 1A,

FIG. 1C is a plot of light transmissivity exhibited by one exemplary implementation of the IOL of FIGS. 1A and 1B for wavelengths in the blue region as a function of radial distance from the optical axis,

FIG. 1D is a schematic cross-sectional view of an IOL having a haptic that transmits light according to one embodiment of the invention,

FIG. 1E is a top view of the IOL of FIG. 1A,

FIG. 2 schematically depicts the IOL of FIGS. 1A and 1B implanted in a patient's eye,

FIG. 3 schematically depicts a conventional IOL implanted in a patient's eye, illustrating that some light rays entering the eye at large visual angles miss the IOL's optic and form a secondary peripheral image on the retina,

FIG. 4A is a schematic top view of an IOL in accordance with another embodiment of the invention,

FIG. 4B is a plot of light transmissivity exhibited by one exemplary implementation of the IOL of FIG. 4A for light in the blue region as a function of radial distance from the optical axis,

FIG. 5A is a schematic cross-sectional view of an IOL according to another embodiment of the invention,

FIG. 5B is a schematic top view of the IOL shown in FIG. 5A,

FIG. 5C is a plot of light transmissivity exhibited by one exemplary implementation of the IOL shown in FIGS. 5A and 5B as a function of radial distance from the optical axis,

FIG. 6A is a schematic cross-sectional view of an IOL according to still another embodiment of the invention,

FIG. 6B is a schematic top view of the IOL shown in FIG. 6A, and

FIG. 6C is a plot of light transmissivity exhibited by one exemplary implementation of the IOL shown in FIGS. 6A and 6B as a function of radial distance from the optical axis.

### DETAILED DESCRIPTION

Some embodiments of the present invention are related to intraocular lenses (IOLs) with non-uniform light transmissivity over at least a portion of an optic of the IOL. As utilized herein, the phrase “light transmissivity” is a dimensionless quantity that refers to the fraction of light energy transmitted through a defined region, i.e., the amount of light energy exiting the defined region divided by the amount of energy entering the defined region. For example, the light transmissivity of a central region of a lens can be defined as the fraction of the flux of light energy normally incident upon an entering surface of the central region that exits the central region via an exiting surface. It is understood that light transmissivity can be relative to any defined region such as an entire optic, or a portion of an optic. As well, the light transmissivity can be relative to a designated portion of the electromagnetic spectrum (e.g., visual range, blue light region of the visual range, etc.).

When non-uniform light transmissivity is utilized in an IOL's optic, some advantageous features may be accrued. For example, if an optic includes a central region which has less light transmissivity relative to an annular region surrounding the central region, the opening and closing of the pupil can act in conjunction with the IOL to control the amount of light reaching the retina. For instance, in bright light situations, the pupil of the eye is smaller and much of the light travels through the filtering central region to limit the intensity on the retina. In low light situations, the pupil of the eye is larger, which can allow more light to strike the annular region that has higher light transmissivity.

Another potential example is directed to the use of an optic with a reduced light transmissivity in a peripheral region thereof. It has been discovered that the shadows perceived by some IOL patients can be caused by a double imaging effect when light enters the eye at very large visual angles. More specifically, in many conventional IOLs, most of the light entering the eye is focused by both the cornea and the IOL onto the retina, but some of the peripheral light misses the IOL and it is hence focused only by the cornea. This leads to the formation of a second peripheral image. Although this image can be valuable since it extends the peripheral visual field, in some IOL users it can result in the perception of a shadow-like phenomenon that can be distracting. This is known as negative dysophotopsia.

Dysphotopsia (e.g., negative dysphotopsia) is often observed by patients in only a portion of their field of vision because the nose, cheek and brow block most high angle peripheral light rays—except those entering the eye from the temporal direction. Moreover, because the IOL is typically designed to be affixed by haptics to the interior of the capsular bag, errors in fixation or any asymmetry in the bag itself can exacerbate the problem—especially if the misalignment causes more peripheral temporal light to bypass the IOL optic.

Thus, some embodiments of the present invention can alleviate, and preferably eliminate, the perception of a dark shadow region by utilizing an optic having reduced light transmissivity in its peripheral portion. When such a peripheral portion is oriented to receive light rays that could bypass a typical IOL optic, the light rays can be directed to the retina with reduced intensity so as to inhibit or eliminate the formation of the secondary image—thus alleviating the perception of dark shadow region by the IOL user. In some instances, the transmission of light is still allowed by an IOL in its peripheral region, but the reduced light transmission can hinder or prevent the formation of sharp contrast features. Accordingly, the IOL can be configured to still allow a patient to perceive peripheral visual features, while not making such features so bright as to be overly distracting.

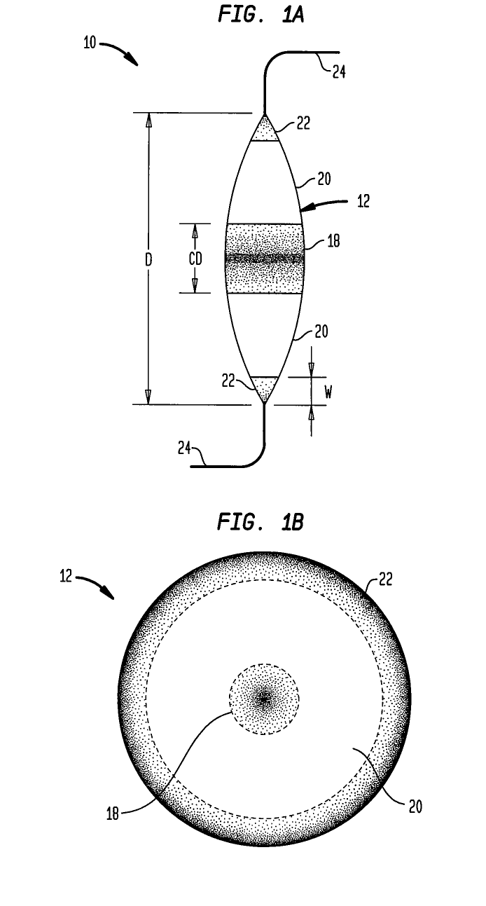

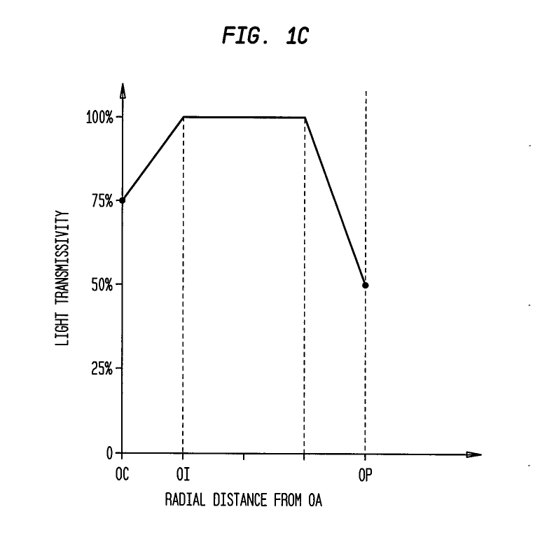

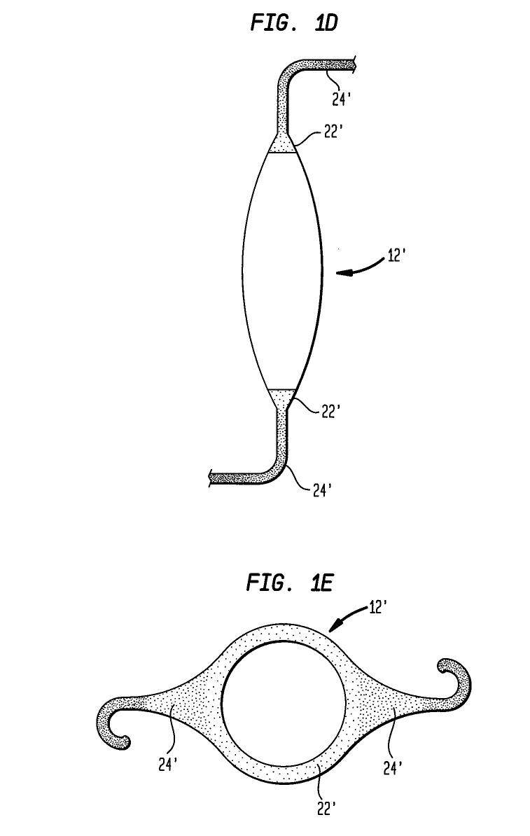

FIGS. 1A and 1B schematically depict an IOL 10 in accordance with one embodiment of the invention that includes an optic 12 having an anterior surface 14 and a posterior surface 16 disposed about an optical axis OA. Although in this exemplary embodiment the optic has a bi-convex shape, in other embodiments it can have other shapes, such as concave-convex, concave-flat, or convex-flat. In many implementations, the optic can provide an optical power in a range of about −15 D to about +34 D.

Although the optic 12 is generally transmissive to visible radiation (e.g., radiation with wavelengths in a range of about 360 nanometers (nm) to about 710 nm), it shows non-uniform light transmissivity across different portions thereof, e.g., the light transmissivity over one portion of the optic differs substantially from the light transmissivity over another portion. In the exemplary embodiment of FIGS. 1A and 1B, the light transmissivity exhibited by the optic for radiation in the blue region of the electromagnetic spectrum having wavelengths in a range of about 400 nm to about 500 nm (corresponding generally to blue light), though rotationally symmetric about the optical axis OA, is radially non-uniform relative to that axis. Providing some filtering of optical radiation in the blue portion of the spectrum before it reaches the retina can be advantageous because of the potential detrimental effects of intense blue light exposure on the retina. The optic 12 can be characterized as having three regions of different light transmissivity: a central region 18, and intermediate region 20 and a peripheral region 22.

More specifically, FIG. 1C shows the optic's light transmissivity within a central region 18 of the optic as a function of radial distance from the optical axis (OA), indicating an increase in light transmissivity from the optic's center (OC) to an intermediate location (OI). The optic's light transmissivity remains substantially constant (e.g., at about 90% in this example) over an intermediate region 18, and exhibits a decrease in a peripheral region 22 of the optic, which extends from an outer boundary of the intermediate location to the optic's periphery (OP). In this embodiment, the increase in light transmissivity from the optic's center to the intermediate location can be characterized by an increasing linear gradient, and its decrease in the peripheral region can be characterized by a decreasing linear gradient. In other embodiments, either gradient can be a non-linear gradient, e.g., one defined by a parabolic function.

As well, each of the regions can be adapted to be characterized by one or more particular values of light transmissivity. For example, the light transmissivity of one or more wavelengths of light (e.g., blue light spectrum) in the center region of an optic 18, as depicted in FIG. 1A, can be no more than about 50 percent, while the light transmissivity in the peripheral region 22 can be no more than about 10 percent (e.g., for light over the entire visible spectrum or just the blue light portion). In another example, the center region 18 and the peripheral region 22 can each have a light transmissivity that is no more than about 50 percent, while the intermediate region 20 can have a light transmissivity no less than about 95 percent (e.g., about 100 percent for visible light). When such values are used to characterize the regions of the optic, the values can refer to a light transmissivity value in the region (e.g., the maximum value), or the average of the transmissivity in the region.

In several embodiments, the optic can exhibit the ability to absorb and/or block the transmission of one or more wavelengths of ultraviolet (herein “UV”) light. Such hindering of UV light transmission can be enhanced relative to a material's inherent UV blocking capabilities (e.g., through the use of additives). While the optic can be configured to allow differing amounts of UV light to be transmitted in different locations (e.g., as depicted and described with respect to FIGS. 1A and 1B) of the optic, in some embodiments the optic uniformly transmits one or more wavelengths of UV light at a reduced level to provide protection to a subject's retina. For example, the optic can be configured to hinder transmittance of at least about 50%, 60%, 70%, 80%, 90%, 95%, or 99% of one wavelength of UV light. Accordingly, the optic can optionally exhibit this blocking/absorption of UV light while also having gradations for changing the transmission of one or more other wavelengths in various portions of an optic.

With continued reference to FIGS. 1A and 1B, in many embodiments, the optic can have a diameter D in a range of about 4 mm to about 9 mm. Further, the optic's central region, which extends from the optic's center to the intermediate location, can have a diameter in a range of about 0.5 mm to about 1 mm, and the peripheral region can have a width (w) in a range of about 0.5 mm to about 1 mm.

With reference to FIG. 1A, the IOL 10 can also include a plurality of fixation members (haptics) that facilitate its placement in the eye. The haptics are formed of a suitable biocompatible polymeric material, such as polymethylmethacrylate. In some embodiments, multipiece IOLs can be formed from separate haptics that are coupled to the optic by employing techniques known in the art. The material from which the haptics are formed can be the same as, or different from, the material forming the optic. It should be appreciated that various haptic designs for maintaining lens stability and centration are known in the art, including, for example, C-loops, J-loops, and plate-shaped haptic designs. Various embodiments of the present invention can be readily employed with these haptic designs.

FIGS. 1D and 1E provide views of other embodiments of an IOLs. In such embodiments, the optic and haptics are made from an integral piece of material (e.g., polymethylmethacrylate). For example, a single piece of material can be milled into the shape of a desired IOL as shown in FIG. 1E. Of course, various other configurations can also be employed.

Single piece construction IOLs can include haptics configured to reduce light transmission of at least one wavelength of light (e.g., blue light between about 500 nm and 600 nm, or between about 400 nm and about 550 nm). As shown in FIG. 1D, the optic 12′ can have peripheral portion 22′ that can decrease light transmissivity for at least one wavelength. In the embodiment shown in FIGS. 1D and 1E, the peripheral portion 22′ has a gradient that decreases the light transmission further as the position moves further from the optic center. In some instances, the haptics 24′ can also exhibit light transmissivity, though hindered for at least one wavelength of visible light. For instance, a haptic can be configured to exhibit non-uniform light transmission (e.g., a gradient of light transmission). The light transmission for one or more wavelengths of light can be continuous with the periphery of the optic at the point of coupling between the optic and haptic. A haptic 24′ with a light transmission gradient can have a gradient that is the same or different from the end portion 22′, or can have a constant light transmission, in one of more portions, at any desired level. IOL's with such haptics can help enhance the ability of the IOL to reduce glare from peripheral rays.

In some embodiments, the haptics can have asymmetric light transmission properties relative to one another. For example, the haptic configured on the nasal side of a patient's head can have a gradient for one or more wavelengths of light, while the haptic on the temporal side can be adapted to have a single level of light transmission, which can be high in some instances (e.g., above about 90%). In another example, the haptic on the temporal side can generally have a lower constant level of light transmissivity relative to the nasal side's haptic. Such configurations can be advantageous since some of the problems from peripheral light rays are generally from rays emanating from the temporal side of a patient's eye. It is understood that such light transmitting haptics need not be necessarily be made integral with the IOL, as separate pieces that are assembled can also be utilized.

The IOL 10 can be implanted in a patient's eye by utilizing surgical techniques known in the art. For example, during cataract surgery, a clouded natural lens can be removed and replaced with the IOL 10. By way of example, an incision can be made in the cornea, e.g., via a diamond blade, to allow other instruments to enter the eye. Subsequently, the anterior lens capsule can be accessed via that incision to be cut in a circular fashion and removed from the eye. A probe can be inserted through the corneal incision to break up the natural lens via ultrasound, and the lens fragments can be aspirated. An injector can be employed to place the IOL, while in a folded state, in the original lens capsule. Upon insertion, the IOL can unfold and its haptics can anchor it within the capsular bag.

In some cases, the IOL is implanted into the eye by utilizing an injector system rather than employing forceps insertion. For example, an injection handpiece having a nozzle adapted for insertion through a small incision into the eye can be used. The IOL can be pushed through the nozzle bore to be delivered to the capsular bag in a folded, twisted, or otherwise compressed state. The use of such an injector system can be advantageous as it allows implanting the IOL through a small incision into the eye, and further minimizes the handling of the IOL by the medical professional. By way of example, U.S. Pat. No. 7,156,854 entitled “Lens Delivery System,” which is herein incorporated by reference, discloses an IOL injector system. The IOLs according to various embodiments of the invention, such as the IOL 10, are preferably designed to inhibit dysphotopsia, e.g., in a manner discussed further below, while ensuring that their shapes and sizes allow them to be inserted into the eye via the injector systems through small incision.

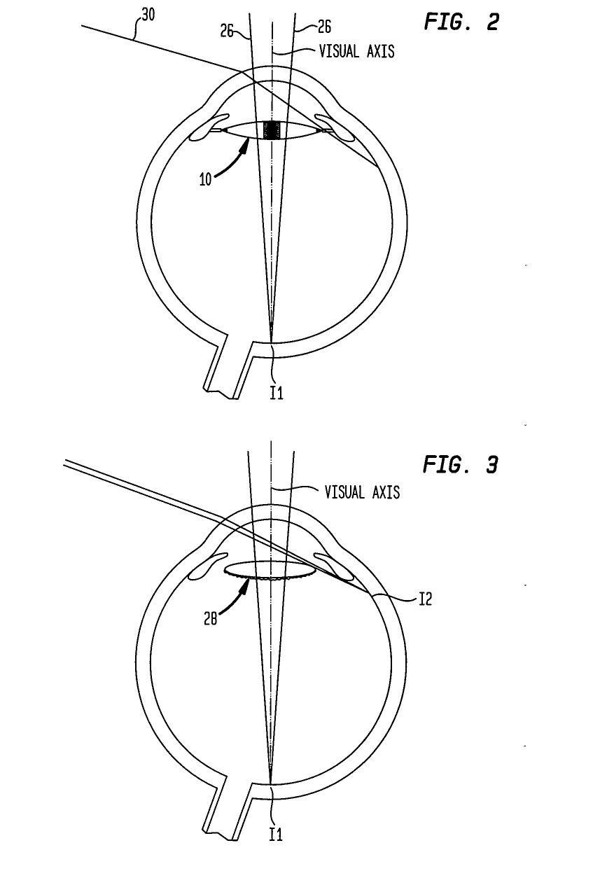

With reference to FIG. 2, once implanted in a patient's eye, the IOL's optic can focus light rays emanating from a field of view (such as exemplary rays 24) to form an image of the field of view on the retina. The optic's central region provides some filtering of the blue light, e.g., due to a lower light transmission in the blue region, to protect the retina from potentially harmful blue rays, especially for small pupil diameters. By way of example, the central region can provide an average filtration of the blue light in a range of about 5 percent to about 95 percent. This filtration, which can optionally include light filtering by an optic's periphery, can result in a change in energy distribution. For example, the filtration of the blue light by the central region, which can have a diameter of about 1 mm, may cause a slight color shift for very small pupil diameters (e.g., pupil diameters less than about 2 mm). In another example, the filtering can result in an intensity shift. As the pupil diameter increases, the filtration of the blue light changes as a function of the pupil diameter. In many implementations, as the pupil diameter increases, no substantial color shift will be observed for average photopic (e.g., pupil diameter of about 3.5 mm) and mesopic (e.g., pupil diameter of about 4.5 mm) conditions.

In some implementations, the low light transmissivity associated with the optic's peripheral region can inhibit visual artifacts that some IOL patients report in their peripheral visual field. By way of illustration and with reference to FIG. 3, when a conventional IOL 28 is implanted in a patient's eye, its optic can form an image 11 of a filed of view. However, some peripheral light rays entering the eye at large visual angles (e.g., angles in a range of about 50 degrees to about 80 degrees relative to the eye's visual axis) may miss the IOL's optic and are hence focused only by the cornea to form a secondary peripheral image 12 that is displaced relative to the primary image II formed by the optic. A retinal region with reduced light intensity (herein also referred to as dark (shadow) region) between these two images can give rise to the perception of a dark shadow by the IOL users. Such dark shadows are generally perceived in only a portion of the field of view (the temporal peripheral visual field) as the nose, cheek, and brow block most high angle peripheral light rays—except those entering from the temporal direction.

In contrast, referring again to FIG. 2, in some implementations in which the IOL 10 is sufficiently large (e.g., it has a diameter greater than about 6 mm such as from about 6 mm to about 9 mm), the optic's peripheral region 22 can receive peripheral light rays entering the eye at large visual angles (such as exemplary light ray 30). As the peripheral region 22 exhibits reduced light transmission, at least with respect to certain wavelengths (e.g., blue light), it reduces the intensity of the light rays passing therethrough to impinge upon the retina. Hence, even if those rays form a secondary peripheral image that is displaced from a primary image formed by the IOL's optic, the intensity of that secondary image would be reduced (e.g., by a factor in a range of about 25% to about 75%), thereby ameliorating and preferably preventing dysphotopsia. In addition, in many cases, the peripheral region can provide some focusing of the peripheral light rays toward the periphery of the primary image, thereby inhibiting the formation of the secondary displaced image. In some instances, it can be desirable to have a secondary image with reduced intensity, as such lower contrast images can allow for improved peripheral vision without the distractions of a high contrast secondary image.

In some implementations in which the peripheral region of the optic is employed to inhibit dysphotopsia while the optic's central region provides filtering of the blue light, the peripheral region can be adapted, e.g., via incorporation of appropriate dyes, to provide filtering of light over a wider wavelength range (e.g., in a range of about 350 nm to about 550 nm). For example, the use of a yellow dye in the optic's central region can be used to absorb blue light, with the blue light transmissivity being a function of the concentration of the yellow dye. Using such dyes or others, the light transmissivity of the IOL's peripheral region can be further reduced to more effectively diminish the intensity of peripheral light rays that are incident on that region.

Other implementations can tailor the wavelength range of light whose transmissivity is altered depending upon the various regions of the IOL. For example, with respect to FIGS. 1A and 1B, the central region 18 can be tailored to alter light transmissivity for wavelengths in a blue region of the electromagnetic spectrum (e.g., about 400 nm to about 500 nm). Thus, potentially detrimental blue light can be diminished from entering the eye during bright light situations. The peripheral region 22, however, can be tailored to alter transmissivity of light having wavelengths across the entire, or a portion, of the visible range (e.g., light with wavelengths less that about 700 nm or in the range of about 400 nm to about 700 nm). Such light alteration by the peripheral region can be beneficial in alleviating dysphotopsia by altering the formation of a secondary image on the retina. It is understood that these exemplary wavelength transmissivity values can be utilized with other embodiments of the invention disclosed herein (e.g., optics can be generally tailored to absorb harmful UV rays). As well, other ranges of wavelengths can also be used consistent with embodiments of the present application.

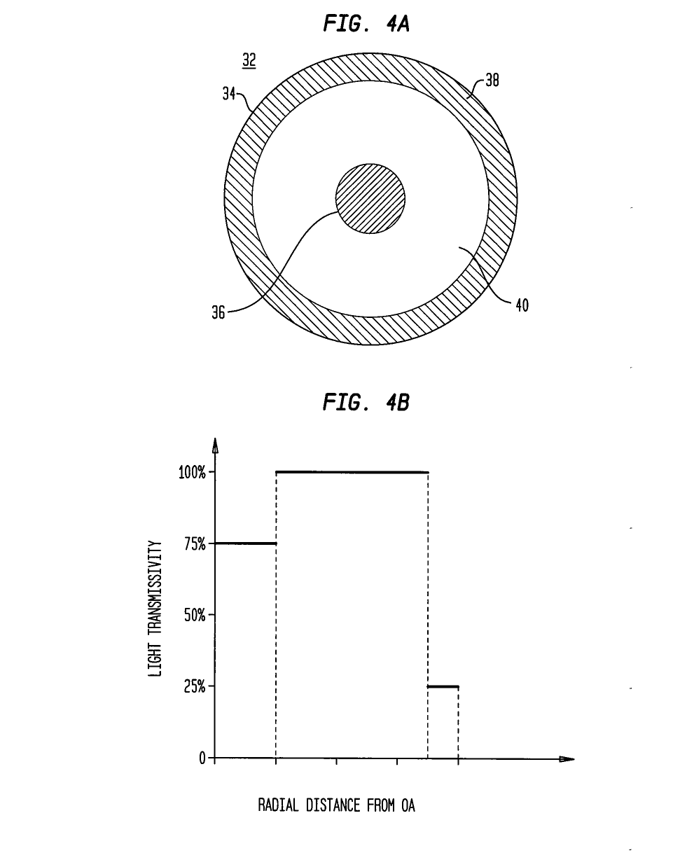

While in the above embodiment the light transmissivity in each of the central and peripheral regions is non-uniform, in other embodiments the light transmissivity in each of those regions can be substantially uniform. By way of example, FIG. 4A schematically depicts an IOL 32 according to another embodiment having an optic 34 that exhibits a non-uniform light transmissivity across different regions thereof. Similar to the previous embodiment, the optic 34 can have a diameter in a range of about 4 mm to about 9 mm. The optic 34 includes a central region 36, e.g., one having a diameter in a range of about 0.5 mm to about 1 mm, and a peripheral region 38, e.g., one having a diameter in a range of about 0.5 mm to about 1 mm, that exhibit a lower light transmissivity for radiation in a given wavelength range (e.g., wavelengths in the blue region of the spectrum) relative an intermediate region 40. The light transmissivity within each region is, however, substantially uniform. By way of further illustration, FIG. 4B presents a plot of the light transmissivity of an exemplary implementation of the optic 34 for a given wavelength band (e.g., the blue region) as a function of radial distance from the optical axis.

Referring again to FIG. 4A, similar to the previous embodiment, the reduced light transmissivity of the optic's central region 36 for blue light can protect the retina against potentially harmful blue light rays. Further, in some implementations, the reduced light transmissivity associated with the optic's peripheral region can ameliorate, and preferably prevent, dysphotopsia.

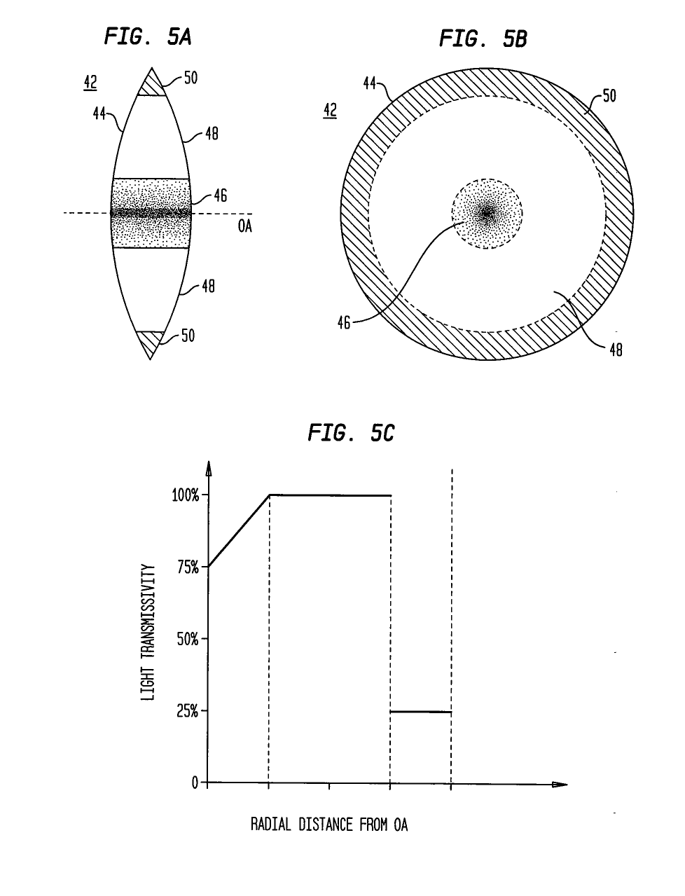

In other embodiments in which the IOL's central and the peripheral regions exhibit reduced light transmissivity relative to its intermediate region, the light transmissivity in at least one of central or the peripheral region can be substantially uniform while in another region it can be characterized by a gradient. For example, FIG. 5 schematically depicts an IOL 42 in accordance with such an embodiment that includes an optic 44 disposed about an optical axis OA, which can be characterized as having a central region 46, an intermediate region 48 and a peripheral region 50. Both the central and the peripheral regions exhibit a reduced light transmissivity for radiation having wavelengths in a given range (e.g., radiation with wavelengths in the blue region of the electromagnetic spectrum) relative to the intermediate region. For example, with reference to FIG. 5C, in this embodiment, the light transmissivity in the radial direction within the central region is characterized by an increasing gradient as a function of increasing distance from the optical axis OA. In contrast, the light transmissivity in the peripheral region is substantially uniform with a value less than the lowest light transmissivity in the central region.

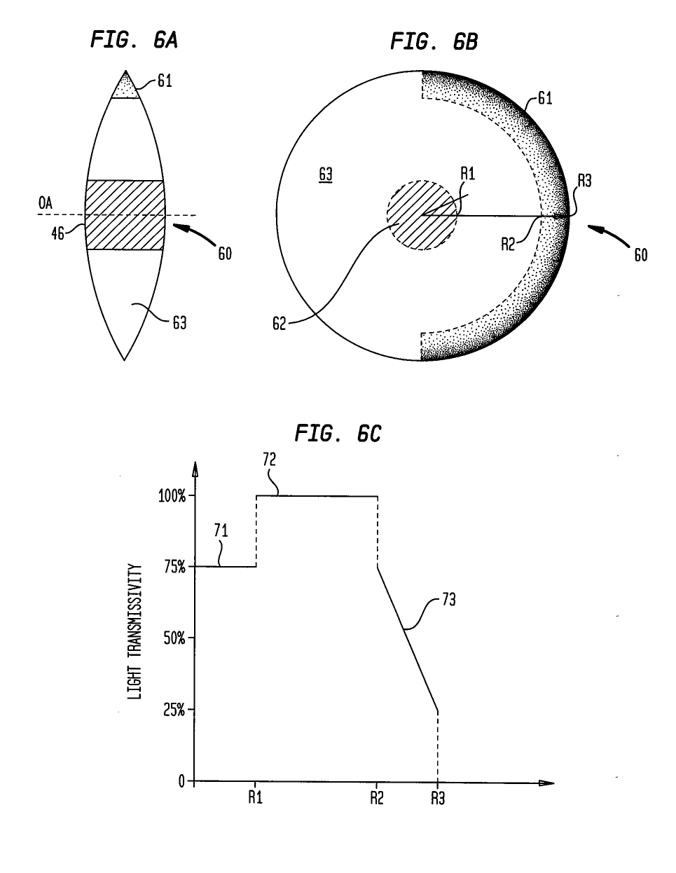

Another embodiment of an IOL is exemplified by FIGS. 6A-6C. As depicted in FIGS. 6A and 6B, an IOL 60 includes a central region 62 and a peripheral region 61, each of which exhibits a lower light transmissivity relative to an intermediate region of the optic 63 that extends between the central and the peripheral regions. In this embodiment, the peripheral region 61 surrounds the intermediate region partially. Accordingly, in some implementations, the IOL 60 can be adapted such that upon its implantation in the eye, the peripheral region 61 would be positioned adjacent to a nasal side of the eye, i.e., opposite the temporal side. Such positioning of the peripheral region can be advantageous since peripheral light rays entering the eye from a temporal direction can be intercepted by the peripheral region 61 and be reduced in intensity and/or redirected so as to inhibit negative dysphotopsia. As well, rather than treating an entire peripheral region of an IOL to provide light filtering, only a portion thereof can be so treated without degrading the optic's ability to inhibit dysphotopsia.

The embodiment of FIGS. 6A-6C also shows that different regions of the optic can exhibit a variety of light transmissivity profiles. As shown in the graph of FIG. 6C, the light transmissivity T1 in the center region 62 is constant from the optical axis OA out to a radial distance R1, albeit at a reduced level relative to that in the intermediate region 63. In the intermediate region 63 between R1 and R2, the light transmissivity T2 is substantially 100%. Finally, the peripheral region 61 between R2 and R3 shows a gradient of decreasing light transmissivity out to the edge of the optic.

In the above embodiments, the IOL optic is preferably formed of a biocompatible material, such as soft acrylic, silicone, hydrogel, or other biocompatible polymeric materials having a requisite index of refraction. By way of example, in some embodiments, the optic can be formed of a cross-linked copolymer of 2-phenylethyl acrylate and 2-phenylethyl methacrylate, which is commonly known as Acrysof®. In many implementations, the biocompatible polymeric material of the optic can be impregnated non-uniformly with one or more dyes to impart a non-uniform light transmissivity to the optic. Some examples of such dyes are provided in U.S. Pat. Nos. 5,528,322 (entitled “Polymerizable Yellow Dyes And Their Use In Ophthalmic Lenses”), 5,470,932 (entitled “Polymerizable Yellow Dyes And Their Use In Ophthalmic Lenses”), 5,543,504 (entitled “Polymerizable Yellow Dyes And Their Use In Ophthalmic Lenses), and 5,662,707 (entitled “Polymerizable Yellow Dyes And Their Use In Ophthalmic Lenses), all of which are herein incorporated by reference.

Further, the IOL's fixation members can be formed of suitable biocompatible materials, such as polymethylmethacrylate (PMMA).

In some cases, the fabrication of an optic exhibiting non-uniform transmissivity can include casting one or more pellets providing light filtration within a biocompatible material. By way of example, such a pellet can have a graduated thickness as a function of diameter in order to tailor light transmission as a function of pupil diameter. Alternatively, one or more pellets having uniform diameters can be employed.

Those having ordinary skill in the art will appreciate that various changes can be made to the above embodiments without departing from the scope of the present invention.

1. An intraocular lens (IOL) comprising:
an optic for implantation in a subject's eye; the optic exhibiting non-uniform light transmissivity over at least a portion of the optic so as to inhibit perception of visual artifacts in a peripheral visual field of the subject's eye.

2. The IOL of claim 1, wherein the optic comprises a peripheral region exhibiting reduced light transmissivity, in at least a segment of the peripheral region.

3. The IOL of claim 1, further comprising:
at least one haptic coupled to the peripheral region of the optic, the at least one haptic exhibiting light transmissivity, the light transmissivity being less than 100%.

4. The IOL of claim 3, wherein the optic and the at least one haptic are formed from an integral piece of material.

5. The IOL of claim 2, wherein the segment with reduced light transmissivity is positioned on the nasal side of the eye once the IOL is implanted in the eye.

6. The IOL of claim 1, wherein the optic exhibits non-uniform light transmissivity for at least one visible wavelength in a blue portion of the visible electromagnetic spectrum.

7. The IOL of claim 6, wherein the optic exhibits hindered light transmissivity for at least one wavelength of light in the ultraviolet range.

8. The IOL of claim 1, wherein the optic exhibits hindered light transmissivity for at least one wavelength of light in the ultraviolet range.

9. The IOL of claim 1, wherein the non-uniform light transmissivity is characterized by a center region of the optic having a higher light transmissivity relative to a peripheral region of the optic.

10. The IOL of claim 9, wherein a light transmissivity of at least one wavelength at the center region of the optic is no more than about 50 percent and a light transmissivity of the at least one wavelength at the peripheral region of the optic is no more than about 10 percent.

11. The IOL of claim 9, wherein the non-uniform light transmissivity is characterized by any of a linear or non-linear gradient in at least one of the center region and the peripheral region.

12. The IOL of claim 9, wherein a light transmissivity in the center region of the optic is characterized by filtering at least one wavelength below about 500 nanometers and a light transmissivity in the peripheral region of the optic is characterized by filtering at least one wavelength below about 700 nanometers.

13. The IOL of claim 1, wherein the non-uniform light transmissivity is characterized by an increase in light transmissivity from a center of the optic to an intermediacy of the optic, and is further characterized by a decrease in light transmissivity from the intermediacy of the optic to the periphery of the optic.

14. The IOL of claim 13, wherein the light transmissivity from the center to the intermediacy is characterized by filtering at least one wavelength in a range from about 400 nm to about 500 nm, and the light transmissivity from the intermediary to the periphery is characterized by filtering at least one wavelength in the visible spectrum.

15. The IOL of claim 13, wherein the increase in light transmissivity is characterized by a first gradient, and the decrease in light transmissivity is characterized by a second gradient, each gradient being independently at least one of a linear gradient and a non-linear gradient.

16. The IOL of claim 15, wherein light transmissivities of the first gradient and second gradient characterized by at least one value of less than about 50 percent and light transmissivities in the intermediacy of the optic are characterized by at least one value greater than about 95 percent.

17. The IOL of claim 1, wherein the optic further comprises at least one dye adapted such that a portion of the optic has at least one selected light transmissivity.

18. An intraocular lens (IOL) comprising:
an optic for implantation in a subject's eye, the optic exhibiting non-uniform light transmissivity such that light transmissivity in an inner region of the optic is greater than light transmissivity in an outer region of the optic.

19. The IOL of claim 18, wherein at least a section of the outer region of the optic with reduced light transmissivity relative to another portion of the optic is positioned on the nasal side of the eye once the IOL is implanted in the eye.

20. The IOL of claim 18, further comprising:
at least one haptic coupled to the outer region of the optic on the nasal side of the eye upon IOL implantation, the at least one haptic exhibiting a light transmissivity for the at least one visible wavelength, the light transmissivity being less than 100%.

21. The IOL of claim 18, wherein the optic exhibits non-uniform light transmissivity for at least one visible wavelength in a blue portion of the visible electromagnetic spectrum.

22. The IOL of claim 21, wherein the optic exhibits hindered light transmissivity for at least one wavelength of light in the ultraviolet range.

23. The IOL of claim 18, wherein the optic exhibits hindered light transmissivity for at least one wavelength of light in the ultraviolet range.

24. The IOL of claim 18, wherein light transmissivity in each of the inner region and the outer region is independently characterized by at least one of a linear gradient and a non-linear gradient.

25. The IOL of claim 24, wherein light transmissivity in the inner region is characterized by filtering at least one wavelength below about 500 nanometers, and light transmissivity in the outer region is characterized by filtering at least one wavelengths below about 700 nanometers.

26. An intraocular lens comprising:
an optic for implantation in a subject's eye, the optic exhibiting non-uniform light transmissivity such that light transmissivity in an inner region of the optic is less than light transmissivity in an intermediate region of the optic, and light transmissivity in an outer region of the optic is less than light transmissivity in the intermediate region.

27. The IOL of claim 26, wherein at least a segment of the outer region of the optic with reduced light transmissivity relative to another portion of the optic is positioned on the nasal side of the eye once the IOL is implanted in the eye.

28. The IOL of claim 26, wherein the optic exhibits non-uniform light transmissivity for at least one visible wavelength in a blue portion of the visible electromagnetic spectrum.

29. The IOL of claim 26, wherein the optic exhibits hindered light transmissivity for at least one wavelength of light in the ultraviolet range.

30. The IOL of claim 26, wherein the lens includes a first gradient in light transmissivity and a second gradient in light transmissivity, each gradient being independently characterized by at least one of a linear function and a non-linear function.

31. The IOL of claim 30, wherein light transmissivities in the first gradient are characterized by filtering at least one wavelength below about 500 nanometers, and light transmissivities in the second gradient are characterized by filtering at least one wavelength of below 700 nanometers.

32. An intraocular lens (IOL), comprising
an optic disposed about an optical axis, the optic exhibiting light transmissivity that is symmetric about the optical axis and radially non-uniform relative to the optical axis, the radial non-uniformity being adapted to inhibit perception of peripheral visual artifacts.

33. The IOL of claim 32, wherein the radial non-uniformity is characterized by a center region of the optic having higher light transmissivity than a peripheral region of the optic.

34. The IOL of claim 33, wherein a light transmissivity of at least one wavelength in the center region of the optic is no more than about 50 percent, and a light transmissivity of the at least one wavelength in the peripheral region of the optic is no more than about 10 percent.

35. The IOL of claim 32, wherein the radial non-uniformity is characterized by an increase in light transmissivity from a center of the optic to an intermediacy of the optic, and is further characterized by a decrease in light transmissivity from the intermediacy of the optic to a periphery of the optic.

36. The IOL of claim 35, wherein light transmissivity of at least one wavelength at the center of the optic are each no more than about 50 percent, and light transmissivity at the intermediacy of the optic is no less than about 95 percent.

37. The IOL of claim 35, wherein the increase in light transmissivity is characterized by a first gradient and the decrease in light transmissivity is characterized by a second gradient, each gradient being independently characterized by at least one of a linear gradient and a non-linear gradient.

38. The IOL of claim 37, wherein light transmissivity in the first gradient is characterized by filtering at least one wavelength below about 500 nanometers, and light transmissivity in the second gradient is characterized by filtering at least one wavelength below about 700 nanometers.

39. The IOL of claim 32, wherein the optic further comprises at least one dye adapted such that a portion of the optic has at least one selected light transmissivity.

40. A method for inhibiting dysphotopsia in a patient having an implanted intraocular lens (IOL), comprising:
directing peripheral light rays intercepted by the IOL such as to inhibit perception of visual artifacts in a peripheral visual field of an eye of the patient, the IOL having non-uniform light transmissivity.

41. The method of claim 40, wherein the IOL exhibits different light transmissivities between a peripheral region of the IOL and another portion of the IOL.

42. The method of claim 41, wherein a light transmissivity in the peripheral region of the IOL is lower than a light transmissivity in the another portion of the IOL.

43. The method of claim 40, wherein the peripheral light rays enter the eye from a temporal side.
